(19) United States
(12) Patent Application Publication
(10) Pub. No.: US 2014/0004512 A1
(43) Pub. Date: Jan. 2, 2014

(54) QUALITY DETERMINATION OF STEM CELLS

(71) Applicant: Simeon Santourlidis, Neuss (DE)

(72) Inventor: Simeon Santourlidis, Neuss (DE)

(73) Assignees: Peter Wernet, Düsseldorf (DE); Foued Ghanjati, Düsseldorf (DE); Simeon Santourlidis, Neuss (DE); Nicole Groth, Grefrath (DE)

(21) Appl. No.: 13/878,915

(22) PCT Filed: Oct. 11, 2011
(86) PCT No.: PCT/EP11/67697

§ 371 Date: Sep. 18, 2013

(30) Foreign Application Priority Data
Oct. 11, 2010 (EP) 10187137.4

(51) Int. Cl. C12Q 1/68

(52) U.S. Cl. CPC C12Q 1/6888
USPC 435/6.11

(57) ABSTRACT

The invention relates to a method for determining the quality of a pluripotent stem cell, comprising the following steps: measuring the DNA methylation of at least one CpG in a CpG island in at least two genes of the pluripotent stem cell, comparing it to the DNA methylation of the at least one CpG in a CpG island in the at least two genes of at least one reference cell, wherein the genes are located on different chromosomes and belong to the gene family of olfactory receptor genes.

The present invention relates to a method for determining the quality of pluripotent stem cells.

In regenerative medicine, a wide variety of approaches are intensively pursued to be able to administer stem cells to patients suffering from degenerative diseases in applications of cell replacement therapy. Already now, therapies using stem cells serving for cartilage regeneration are being performed in various hospitals. In the foreseeable future, the application of stem cells will increase exponentially.

The method of reprogramming endogenous terminally differentiated cells by inducing pluripotency will contribute to this development. This circumvents both problems of immune rejection and ethical aspects.

However, in all existing approaches, this requires in vitro cell culturing, for which no uniform standards exist. For application with humans, it is required to determine the performance or quality of the stem cells.

Chuanying Pan et al., J. Genet. Genomics 37 (2010) 241-248, examined the demethylation of the promoters of NANOG and OCT4 in induced pluripotent stem cells of fibroblasts.

Prashant Mali et al., Stem Cells 28 (2010) 713-720, examined the demethylation of induced pluripotent stem cells by means of a HumanMethylation27 BeadChip.

It is the object of the present invention to provide a simple method for determining the performance and quality.

This object is achieved by a method for determining the quality of pluripotent stem cells, comprising the steps of:
measuring the DNA methylation of at least one CpG in a CpG island in at least three genes of the pluripotent stem cell;
comparing with the DNA methylation of said at least one CpG in a CpG island in said at least three genes of at least one reference cell;

wherein said genes are located on different chromosomes and belong to the gene family of olfactory receptor genes.

The manifestation of a specific cell type, for example, of a stem cell, requires cell-type specific gene expression. This requires qualitatively and quantitatively adapted gene regulation.

One of the essential mechanisms for gene regulation is DNA methylation. Approximately 60% of the human genes are influenced by DNA methylation in differentiated cells. In DNA, cytosine that is in the context of a palindromic CpG dinucleotide can have an additional methyl group. Such correspondingly methylated genes are not expressed, or only so in a greatly reduced way.

However, the methylation is not uniformly distributed in the genes throughout the genome, but is increased in so-called CpG islands, which are in the 5′ regions of the genes.

If a CpG island is completely methylated, transcription of the corresponding gene is not possible. In the case of a non-methylation, the gene has transcriptional competence, i.e., it can be transcribed.

However, the CpG dinucleotides that can be methylated are not equivalent for the regulation of the gene to which they belong. The methylation of particular CpGs, which act through their association with target sequences of transcription factors, exert the greatest influence on gene expression. It is assumed that the methylated CpGs influence the binding of transcription factors through their steric position in the major grooves of the DNA. Thus, an incompletely methylated CpG island can result in a transcription block or reduced transcription depending on which CpGs are methylated, but there may also be no repressing influence on transcription at all.

According to the invention, the DNA methylation of at least one CpG in a CpG island in at least three genes of a pluripotent stem cell to be examined is analyzed, i.e., genes are selected, and methylation is measured on the CpG islands known from these genes on at least one position of the CpG island. Preferably, a position in a CpG island that is as relevant as possible to transcription control is measured. This is followed by comparing such methylation with the methylation of a reference cell, in which the same CpG islands in the same genes and the same positions within the CpG islands of the reference cell are compared.

In one embodiment of the invention, the pluripotent stem cell examined may be an induced pluripotent stem cell, in which case a known pluripotent stem cell is suitable as the reference cell.

If the stem cell examined and the reference cell have similar or identical methylation patterns, this is indicative of successful reprogramming and thus a high quality of the induced pluripotent stem cell obtained. Additionally or alternatively, the starting cell, for example, a fibroblast cell, may also be employed as the reference cell. The extent of alteration of the DNA methylation may also be used to conclude the success of induction therefrom, i.e., the more similar the terminally differentiated cell and the presumably induced stem cell are, the worse is the quality of the induced stem cell.

Stem cells are frequently kept in culture for extended periods of time. There are stem cell lines, in part originating from embryonic stem cells, that are kept in culture for years. In such cases, it cannot be excluded that the stem cells got damaged during the culturing, which may remain unnoticed for a long time. In such a case, the method according to the invention can be employed to observe the degree of methylation of one or more CpG islands from two or more genes in the course of a culture, and to conclude an alteration of the cultured cell from the occurrence of differences. In other words, a cell that exhibits a DNA methylation pattern deviating from that of its originally cultured cell has a lesser quality.

According to the invention, it is preferred that not only the DNA methylation of three genes is determined, but that more genes are employed in order to make differences more pronounced. According to the invention, the number of genes may be three, or four, or five, or seven, or ten, or more.

In order to obtain a representative impression of the methylome, genes that are located on different chromosomes are examined, because this gives an improved overview of the situation of the cell.

In many cases, it will be reasonable to examine genes that at least in part belong to one family of genes. In other embodiments, it may also be reasonable to examine ten genes, for example, wherein some genes belong to one family of genes and the other genes belong to another family of genes.

It is important to the method according to the invention that always the degree of methylation only of identical positions in identical CpG islands of identical genes can be compared.

According to the invention, it is preferred that not only a single position is analyzed in each of the measured CpG islands, but that several positions, or if several CpG islands exist, several positions in several CpG islands, are analyzed.

The family of genes that is employed according to the invention is the gene family of olfactory receptor genes (The human olfactory receptor gene family, Malnic B, Godfrey P A, Buck L B. Proc Natl Acad Sci U.S.A. 2004 Feb 24; 101(8): 2584-9. Erratum in: Proc Natl Acad Sci U.S.A. 2004 May 4; 101(18): 7205, and The mouse olfactory receptor gene family. Godfrey P A, Malnic B, Buck L B. Proc Natl Acad Sci U.S.A. 2004 Feb. 17; 101(7): 2156-61. Epub 2004 Feb 9). The olfactory receptor genes are the largest gene family in the human genome (about 1,000 genes). These genes are associated with a CpG island in their 5′ region in embryonic and induced pluripotent stem cells, and this island is densely methylated in both types of stem cells. In contrast, the same CpG islands of the same genes in fibroblasts are essentially non-methylated. Since this difference can be found several times in each chromosome, it represents a pronounced reflection of the DNA methylation of the genome (“methylome”). The particular advantage of the gene family of olfactory receptor genes resides in their large number, being distributed over all chromosomes, and the dense methylation in embryonic and induced pluripotent stem cells. Thus, it is possible to determine methylation on a large number of genomic sites that can be differentially methylated. Preferably, at least 10 sites, more preferably at least 20, at least 50, at least 100 or at least 1000 sites are analyzed and employed for the evaluation.

The nomenclature of the genes is “ORnXm”, where

“OR” represents the olfactory receptor superfamily;

“n” is an integer representing the family, wherein the members have a sequence identity of more than 40%;

“X” is a single letter representing a subfamily, wherein the members have a sequence identity of more than 60%; and

“m” is an integer designating an individual family member.

Thus, for example, OR1A1 is the first isoform of the subfamily A of olfactory receptor family 1.

It is considered that receptors of the same subfamily recognize similar molecules.

There are two large groups, class I (fish-like receptor) with the OR families 51 to 56, and class II (tetrapod) with the OR families 1 to 13.

Preferably used receptor genes are the following: OR1A1; OR1A2; OR1AA1P; OR1AB1P; OR1AC1P; OR1B1; OR1C1; OR1D2; OR1D3P; OR1D4; OR1D5; OR1E1; OR1E2; OR1E3; OR1F1; OR1F2P; OR1F12; OR1G1; OR1H1P; OR1I1; OR1J1; OR1J2; OR1J4; OR1K1; OR1L1; OR1L3; OR1L4; OR1L6; OR1L8; OR1M1; OR1M4P; OR1N1; OR1N2; OR1P1; OR1Q1; OR1R1P; OR1S1; OR1S2; OR1X1P; OR1X5P; OR2A1; OR2A2; OR2A3P; OR2A4; OR2A5; OR2A7; OR2A9P; OR2Al2; OR2A13P; OR2A14; OR2A15P; OR2A20P; OR2A25; OR2A41P; OR2A42; OR2AD1P; OR2AE1; OR2AF1P; OR2AG1; OR2AG2; OR2AH1P; OR2AI1P; OR2A31; OR2AK2; OR2AL1P; OR2AM1P; OR2AO1P; OR2AP1; OR2AQ1P; OR2AS1P; OR2AS2P; OR2AT1P; OR2AT2P; OR2AT4; OR2B2; OR2B3; OR2B4P; OR2B6; OR2B7P; OR2B8P; OR2B11; OR2BH1P; OR2C1; OR2C3; OR2D2; OR2D3; OR2E1P; OR2F1; OR2F2; OR2G1P; OR2G2; OR2G3; OR2G6; OR2H1; OR2H2; OR2H4P; OR2H5P; OR2I1P; OR2J1; OR2J2; OR2J3; OR2J4P; OR2K2; OR2L1P; OR2L2; OR2L3; OR2L5; OR2L6P; OR2L8; OR2L9P; OR2L13; OR2M1P; OR2M2; OR2M3; OR2M4; OR2M5; OR2M7; OR2N1P; OR2P1P; OR2Q1P; OR2R1P; OR2S1P; OR2S2; OR2T1; OR2T2; OR2T3; OR2T4; OR2T5; OR2T6; OR2T7; OR2T8; OR2T10; OR2T11; OR2T12; OR2T27; OR2T29; OR2T32P; OR2T33; OR2T34; OR2T35; OR2U1P; OR2U2P; OR2V1; OR2V2; OR2W1; OR2W2P; OR2W3; OR2W4P; OR2W5; OR2W6P; OR2X1P; OR2Y1; OR2Z1; OR3A1; OR3A2; OR3A3; OR3A4P; OR3B1P; OR3D1P; OR4A1P; OR4A2P; OR4A3P; OR4A4P; OR4A5; OR4A6P; OR4A7P; OR4A8P; OR4A9P; OR4A10P; OR4A11P; OR4Al2P; OR4A13P; OR4A14P; OR4A15; OR4A16; OR4A17P; OR4A18P; OR4A19P; OR4A21P; OR4A40P; OR4A41P; OR4A42P; OR4A43P; OR4A44P; OR4A45P; OR4A46P; OR4A47; OR4A48P; OR4A49P; OR4A50P; OR4B1; OR4B2P; OR4C1P; OR4C2P; OR4C3; OR4C4P; OR4C5; OR4C6; OR4C7P; OR4C9P; OR4C10P; OR4C11; OR4C12; OR4C13; OR4C14P; OR4C15; OR4C16; OR4C45; OR4C46; OR4C48P; OR4C49P; OR4C50P; OR4D1; OR4D2; OR4D5; OR4D6; OR4D7P; OR4D8P; OR4D9; OR4D10; OR4D11; OR4D12P; OR4E1; OR4E2; OR4F1P; OR4F2P; OR4F3; OR4F4; OR4F5; OR4F6; OR4F7P; OR4F8P; OR4F13P; OR4F14P; OR4F15; OR4F16; OR4F17; OR4F21; OR4F28P; OR4F29; OR4G1P; OR4G2P; OR4G3P; OR4G4P; OR4G6P; OR4G11P; OR4H6P; OR4H12P; OR4K1; OR4K2; OR4K3; OR4K4P; OR4K5; OR4K6P; OR4K7P; OR4K8P; OR4K11P; OR4K12P; OR4K13; OR4K14; OR4K15; OR4K16P; OR4K17; OR4L1; OR4M1; OR4M2; OR4N1P; OR4N2; OR4N3P; OR4N4; OR4N5; OR4P1P; OR4P4; OR4Q1P; OR4Q2; OR4Q3; OR4R1P; OR4R2P; OR4R3P; OR4S1; OR4S2; OR4T1P; OR4U1P; OR4V1P; OR4W1P; OR4X1; OR4X2; OR4X7P; OR5A1; OR5A2; OR5AC1; OR5AC2; OR5AC4P; OR5AH1P; OR5AK1P; OR5AK2; OR5AK3P; OR5AK4P; OR5AL1; OR5AL2P; OR5AM1P; OR5AN1; OR5AN2P; OR5AO1P; OR5AP1P; OR5AP2; OR5AQ1P; OR5AR1; OR5AS1; OR5AU1; OR5AW1P; OR5AZ1P; OR5B1P; OR5B2; OR5B3; OR5B10P; OR5B12; OR5B15P; OR5B17; OR5B19P; OR5B21; OR5BA1P; OR5BB1P; OR5BC1P; OR5BD1P; OR5BE1P; OR5BH1P; OR5BJ1P; OR5BK1P; OR5BL1P; OR5BM1P; OR5BN1P; OR5BN2P; OR5BP1P; OR5BQ1P; OR5BR1P; OR5BS1P; OR5BT1P; OR5C1; OR5D2P; OR5D3P; OR5D13; OR5D14; OR5D15P; OR5D16; OR5D17P; OR5D18; OR5E1P; OR5F1; OR5F2P; OR5G1P; OR5G3; OR5G4P; OR5G5P; OR5H1; OR5H2; OR5H3P; OR5H4P; OR5H5P; OR5H6; OR5H7P; OR5H8P; OR5H14; OR5H15; OR5I1; OR5J1P; OR5J2; OR5J7P; OR5K1; OR5K2; OR5K3; OR5K4; OR5L1; OR5L2; OR5M1; OR5M2P; OR5M3; OR5M4P; OR5M5P; OR5M6P; OR5M7P; OR5M8; OR5M9; OR5M10; OR5M11; OR5M12P; OR5M13P; OR5M14P; OR5P1P; OR5P2; OR5P3; OR5P4P; OR5R1; OR5S1P; OR5T1; OR5T2; OR5T3; OR5V1; OR5W1P; OR5W2; OR6A2; OR6B1; OR6B2; OR6B3; OR6C1; OR6C2; OR6C3; OR6C4; OR6C5P; OR6C6; OR6C7P; OR6C64P; OR6C65; OR6C66P; OR6C68; OR6C69P; OR6C70; OR6C71P; OR6C72P; OR6C73P; OR6C74; OR6C75; OR6C76; OR6D1P; OR6E1P; OR6F1; OR6J1; OR6K1P; OR6K2; OR6K3; OR6K4P; OR6K5P; OR6K6; OR6L1P; OR6L2P; OR6M1; OR6M2P; OR6M3P; OR6N1; OR6N2; OR6P1; OR6Q1; OR6R1P; OR6R2P; OR6S1; OR6T1; OR6U2P; OR6V1; OR6W1P; OR6X1; OR6Y1; OR7A1P; OR7A2P; OR7A3P; OR7A5; OR7A8P; OR7A10; OR7A11P; OR7A15P; OR7A17; OR7A18P; OR7A19P; OR7C1; OR7C2; OR7D1P; OR7D2; OR7D4; OR7D11P; OR7E1P; OR7E2P; OR7E4P; OR7E5P; OR7E7P; OR7E8P; OR7E10P; OR7E11P; OR7E12P; OR7E13P; OR7E14P; OR7E15P; OR7E16P; OR7E18P; OR7E19P; OR7E21P; OR7E22P; OR7E23P; OR7E24; OR7E25P; OR7E26P; OR7E28P; OR7E29P; OR7E31P; OR7E33P; OR7E35P; OR7E36P; OR7E37P; OR7E38P; OR7E39P; OR7E41P; OR7E43P; OR7E46P; OR7E47P; OR7E53P; OR7E55P; OR7E59P; OR7E62P; OR7E66P; OR7E83P; OR7E84P; OR7E85P; OR7E86P; OR7E87P; OR7E89P; OR7E90P; OR7E91P; OR7E93P; OR7E94P; OR7E96P; OR7E97P; OR7E99P; OR7E100P; OR7E101P; OR7E102P; OR7E104P; OR7E105P; OR7E106P; OR7E108P; OR7E109P; OR7E110P; OR7E111P; OR7E115P; OR7E116P; OR7E117P; OR7E121P; OR7E122P; OR7E125P; OR7E126P; OR7E128P; OR7E129P; OR7E130P; OR7E136P; OR7E140P; OR7E145P; OR7E148P; OR7E149P; OR7E154P; OR7E155P; OR7E156P; OR7E157P; OR7E158P; OR7E159P; OR7E160P; OR7E161P; OR7E162P; OR7G1; OR7G2; OR7G3; OR7G15P; OR7H1P; OR7H2P; OR7K1P; OR7L1P; OR7M1P; OR8A1; OR8A2P; OR8A3P; OR8B1P; OR8B2; OR8B3; OR8B4; OR8B5P; OR8B6P; OR8B7P; OR8B8; OR8B9P; OR8B10P; OR8B12; OR8C1P; OR8D1; OR8D2; OR8D4; OR8F1P; OR8G1; OR8G2; OR8G3P; OR8G5; OR8G7P; OR8H1; OR8H2; OR8H3; OR8I1P; OR812; OR8I4P; OR8J1; ORM; ORM; OR8K1; OR8K2P; OR8K3; OR8K4P; OR8K5; OR8L1P; OR8Q1P; OR8R1P; OR8S1; OR8S21P; OR8T1P; OR8U1; OR8U8; OR8U9; OR8V1P; OR8X1P; OR9A1P; OR9A2; OR9A3P; OR9A4; OR9G1; OR9G2P; OR9G3P; OR9G4; OR9G9; OR9H1P; OR911; OR9I2P; OR9I3P; OR9K1P; OR9K2; OR9L1P; OR9M1P; OR9N1P; OR9P1P; OR9Q1; OR9Q2; OR9R1P; OR9S24P; OR10A2; OR10A3; OR10A4; OR10A5; OR10A6; OR10A7; OR10AA1P; OR10AB1P; OR10AC1P; OR10AD1; OR10AE1P; OR10AE3P; OR10AF1P; OR10AG1; OR10AH1P; OR10AK1P; OR10B1P; OR10C1; OR10D1P; OR10D3; OR10D4P; OR10D5P; OR10G1P; OR10G2; OR10G3; OR10G4; OR10G5P; OR10G6; OR10G7; OR10G8; OR10G9; OR1OH1; OR10H2; OR10H3; OR10H4; OR10H5; OR10J1; OR10J2P; OR10J3; OR10J4; OR10J5; OR10J6P; OR10J7P; OR10J8P; OR10J9P; OR10K1; OR10K2; OR1ON1P; OR10P1; OR10Q1; OR10Q2P; OR10R1P; OR10R2; OR10R3P; OR10S1; OR10T1P; OR10T2; OR10U1P; OR10V1; OR10V2P; OR10V3P; OR10V7P; OR10W1; OR10X1; OR10Y1P; OR10Z1; OR11A1; OR11G1P; OR11G2; OR11H1; OR11H2; OR11H3P; OR11H4; OR11H5P; OR11H6; OR11H7; OR11H12; OR11H13P; OR11I1P; OR11J1P; OR11J2P; OR11J5P; OR11K1P; OR11K2P; OR11L1; OR11M1P; OR11N1P; OR11P1P; OR11Q1P; OR12D1P; OR12D2; OR12D3; OR13A1; OR13C1P; OR13C2; OR13C3; OR13C4; OR13C5; OR13C6P; OR13C7P; OR13C8; OR13C9; OR13D1; OR13D2P; OR13D3P; OR13E1P; OR13F1; OR13G1; OR13H1; OR13I1P; OR13J1; OR13K1P; OR13Z1P; OR13Z2P; OR13Z3P; OR14A2; OR14A16; OR14C36; OR1411; OR14J1; OR14K1; OR14L1P; OR51A1P; OR51A2; OR51A3P; OR51A4; OR51A5P; OR51A6P; OR51A7; OR51A8P; OR51A9P; OR51A10P; OR51AB1P; OR51B2; OR51B3P; OR51B4; OR51B5; OR51B6; OR51B8P; OR51C1P; OR51C4P; OR51D1; OR51E1; OR51E2; OR51F1; OR51F2; OR51F3P; OR51F4P; OR51F5P; OR51G1; OR51G2; OR51H1P; OR51H2P; OR5111; OR5112; OR51J1; OR51K1P; OR51L1; OR51M1; OR51N1P; OR51P1P; OR51Q1; OR51R1P; OR51S1; OR51T1; OR51V1; OR52A1; OR52A4; OR52A5; OR52B1P; OR52B2; OR52B3P; OR52B4; OR52B5P; OR52B6; OR52D1; OR52E1; OR52E2; OR52E3P; OR52E4; OR52E5; OR52E6; OR52E7P; OR52E8; OR52H1; OR52H2P; OR5211; OR5212; OR52J1P; OR52J2P; OR52J3; OR52K1; OR52K2; OR52K3P; OR52L1; OR52L2P; OR52M1; OR52M2P; OR52N1; OR52N2; OR52N3P; OR52N4; OR52N5; OR52P1P; OR52P2P; OR52Q1P; OR52R1; OR52S1P; OR52T1P; OR52U1P; OR52V1P; OR52W1; OR52X1P; OR52Y1P; OR52Z1; OR55B1P; OR56A1; OR56A3; OR56A4; OR56A5; OR56A7P; OR56B1; OR56B2P; OR56B3P; OR56B4.

A wide variety of methods are commonly used as methods for determining DNA methylation. One common method is methylation-specific PCR. In this method, methylated cytosine is converted to uracil using bisulfite. By using specific primers, it can be examined whether or not the sites to be examined are methylated. The measuring method is a real time PCR in which the labeled marker or labeled probes are employed.

An alternative determining method is the so-called Nimble-Gene by the Roche company. In this method, DNA fragments are precipitated by means of 5′-methylcytidine-specific antibodies, isolated and detected after amplification on an array.

Other methods, for example, by radioactive labeling, Southern blotting or the like, are also possible.

When several CpG islands or several positions in one CpG island or several genes are analyzed, the methylation-specific PCR reactions employed can also be performed in one PCR with a large number of primers in principle. In order to increase the specificity of PCR, it is preferred that the respective examinations are performed separately.

In many embodiments, it will be helpful to additionally introduce controls in order to check the quality of the measurement.

Since differences may occur between the measurements, it will also be often reasonable to check the reference cell and the stem cell simultaneously in two reactions. However, in many cases, it will suffice to recur to earlier measurements and correspondingly stored data for the methylation of the stem cells.

The invention also relates to a method for determining the quality of a pluripotent stem cell, comprising the steps of:
measuring the DNA methylation of at least one CpG in a CpG island in at least two genes of the pluripotent stem cell;
comparing with the DNA methylation of said at least one CpG in a CpG island in said at least two genes of at least one reference cell.

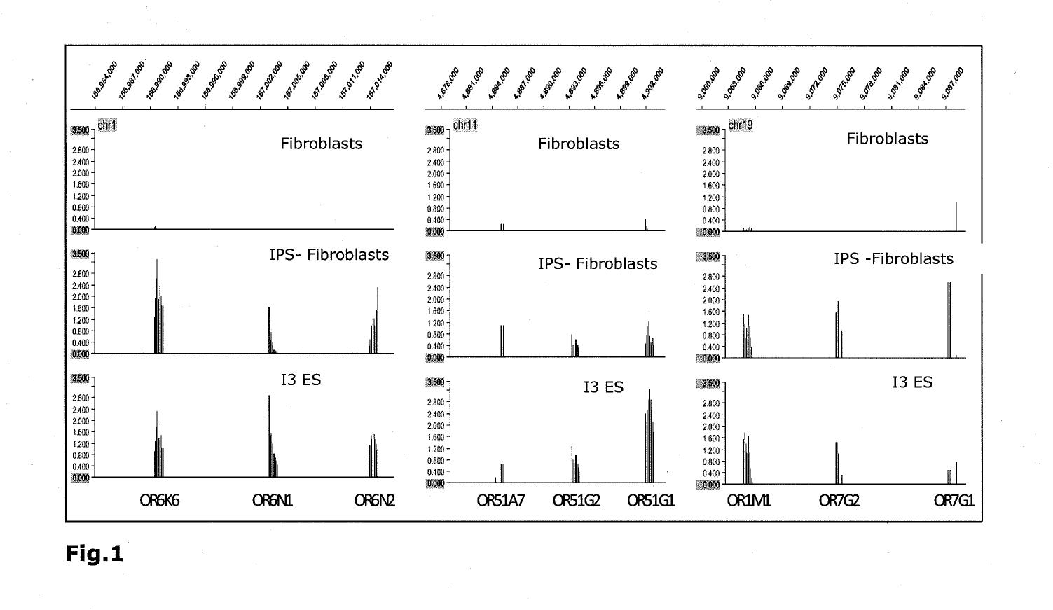

FIG. 1 shows the DNA methylation status in the 5′ region of three olfactory receptor genes each on three different chromosomes in fibroblasts, IPS fibroblasts and ES cells (I3).

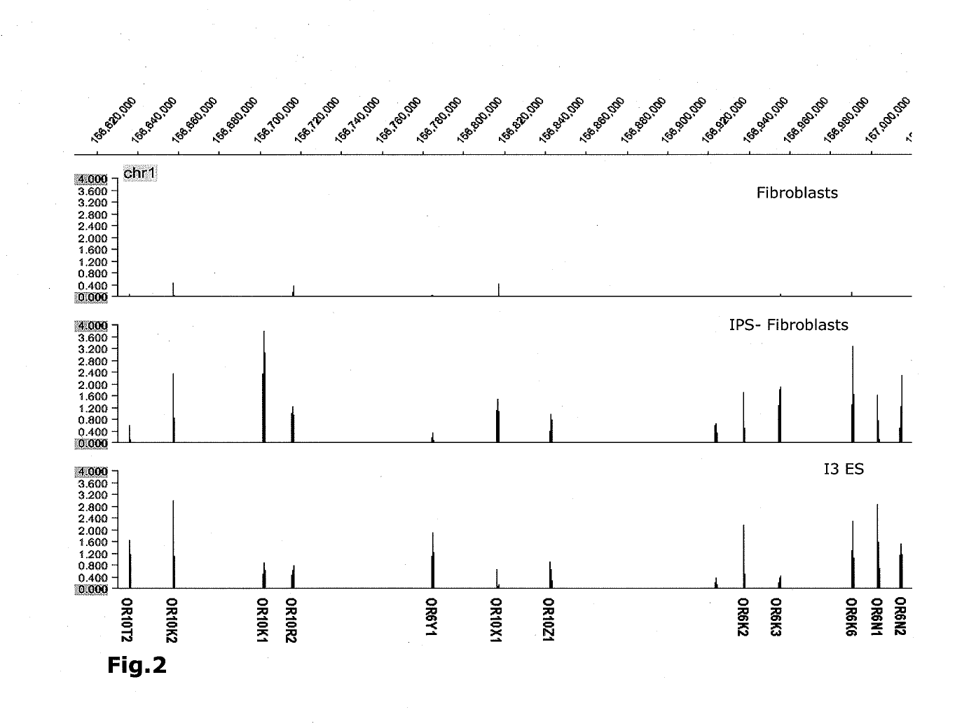

FIG. 2 shows the DNA methylation status in the 5′ region of olfactory receptor genes on chromosome 1 in fibroblasts, IPS fibroblasts and ES cells (I3).

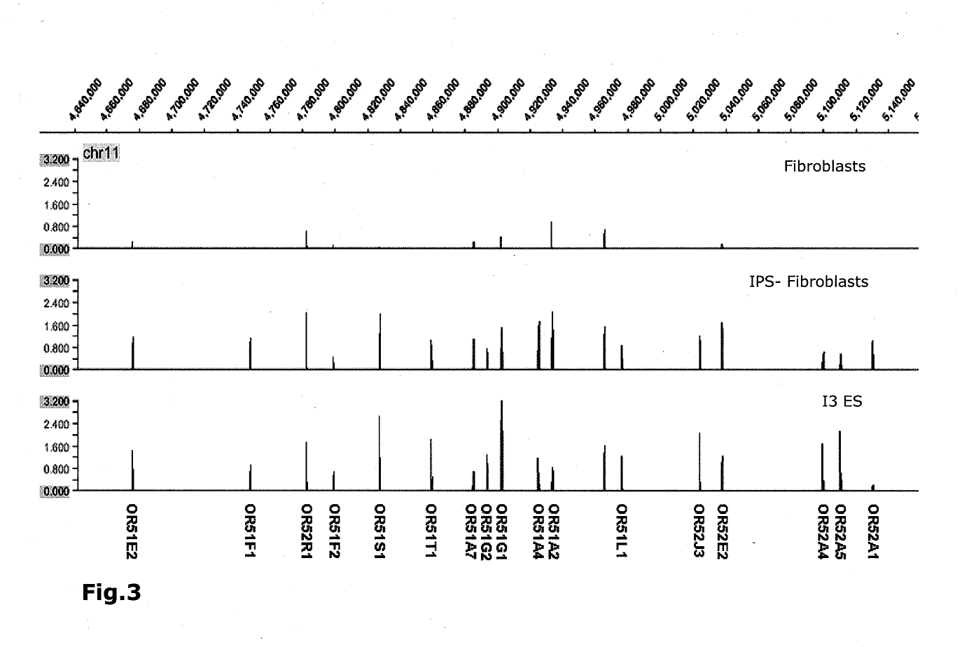

FIG. 3 shows the DNA methylation status in the 5′ region of olfactory receptor genes on chromosome 11 in fibroblasts, IPS fibroblasts and ES cells (I3).

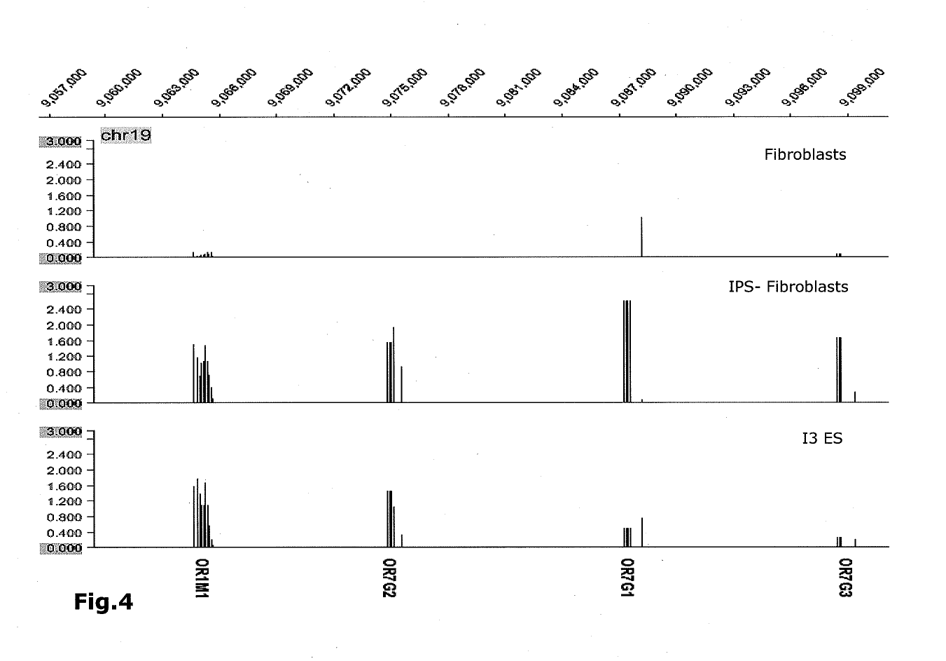

FIG. 4 shows the DNA methylation status in the 5′ region of olfactory receptor genes on chromosome 19 in fibroblasts, IPS fibroblasts and ES cells (I3).

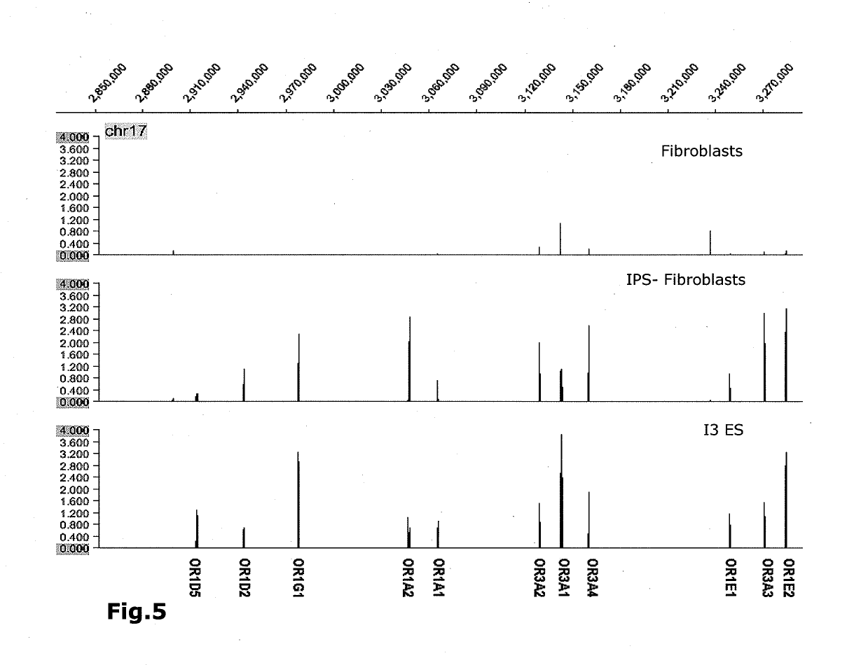

FIG. 5 shows the DNA methylation status in the 5′ region of olfactory receptor genes on chromosome 17 in fibroblasts, IPS fibroblasts and ES cells (I3).

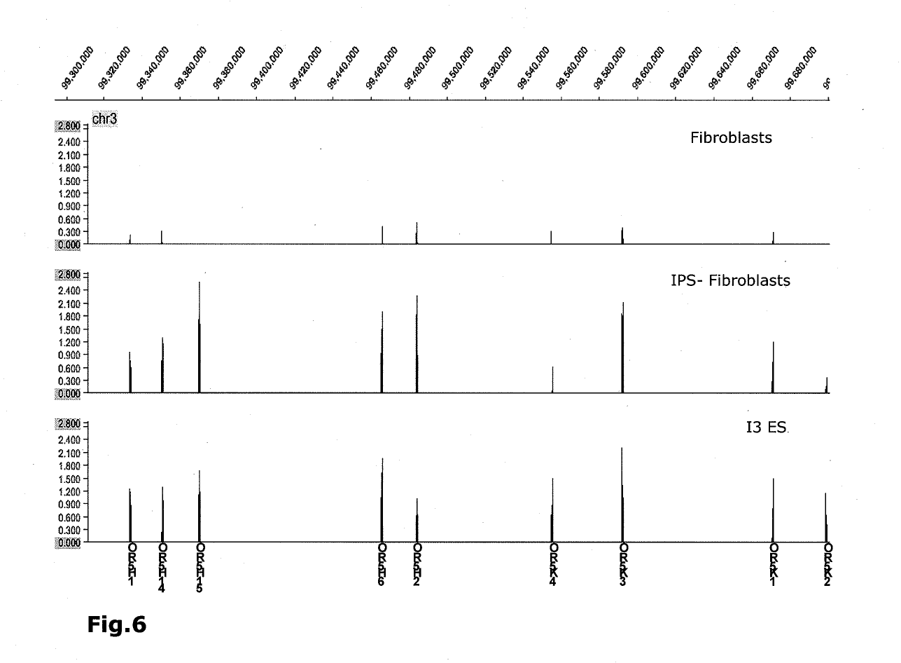

FIG. 6 shows the DNA methylation status in the 5′ region of olfactory receptor genes on chromosome 3 in fibroblasts, IPS fibroblasts and ES cells (I3).

The invention is further illustrated by the following Examples:

### 1. Isolation of Genomic DNA

Genomic DNA was isolated as follows: The genomic DNA was isolated from the cells by means of the Qiagen DNeasy blood&tissue DNA isolations kit.

### 2. Isolation of Methylated DNA

1 μg of genomic DNA was converted by ultrasonication to a fragment size of about 300 to 1,000 base pairs. The methylated DNA fragments were precipitated by means of a methylcytosine-specific antibody; a Methylamp Methylated DNA Capture Kit (MeDIP from Diagenode) was used.

### 3. Analysis of Methylation

The precipitates were hybridized on a Nimble-Gene 385K Ref. Seq. Promoter Array HG18 (Roche). The promoter regions of all known genes rich in CpG dinucleotides are present on this array, covalently bonded in the form of 50 mer oligonucleotide samples.

The hybridized arrays were scanned with a microarray scanner (Molecular Devices), and images were generated with the Axon Genepix software. Nimble-Scan Version 2.5 and Signal-Map Version 1.9 were employed for analysis.

### 4. Determination of Quality

The cells employed were cells obtained from (a) fibroblasts, (b) pluripotent stem cells induced from fibroblasts by retroviral transfer of the four transcription factors Oct3/4, Sox2, c-Myc, and Klf4 (by analogy with: Induction of pluripotent stem cells from mouse embryonic and adult fibroblast cultures by defined factors. Takahashi K, Yamanaka S. Cell. 2006 Aug. 25; 126(4): 663-76. Epub 2006 Aug. 10), and (c) a known embryonic cell line 1-3. CpG methylation on olfactory receptor genes from different chromosomes was analyzed. The results of the analyses are shown in FIGS. 1 to 6.

1. A method for determining the quality of a pluripotent stem cell, comprising the steps of:
measuring the DNA methylation of at least one CpG in a CpG island in at least three genes of the pluripotent stem cell;
comparing with the DNA methylation of said at least one CpG in a CpG island in said at least three genes of at least one reference cell;
wherein said genes are located on different chromosomes and belong to the gene family of olfactory receptor genes.

2. The method according to claim 1, wherein said pluripotent stem cell is an induced pluripotent stem cell.

3. The method according to claim 1, wherein said reference cell is a pluripotent stem cell.

4. The method according to claim 1, wherein said reference cell is a differentiated cell.

5. The method according to claim 1, wherein at least two reference cells are employed.

6. The method according to claim 1, wherein the methylation of at least one CpG island each in at least four or at least five genes of said pluripotent stem cell is measured and respectively compared with the methylation in genes of the reference cells.

7. The method according to claim 1, wherein said olfactory receptor genes are human members of class I or II.

8. The method according to claim 1, wherein several CpG islands are measured in each gene.

9. The method according to claim 1, wherein the methylation of at least one CpG island each in at least seven or at least ten genes of said pluripotent stem cell is measured and respectively compared with the methylation in genes of the reference cells.

10. The method according to claim 1, wherein said olfactory receptor genes are selected from OR6K6, OR6N1, OR6N2, OR51A7, OR51G2, OR51G1, OR1M1, OR7G2, OR7G1.

11. The method according to claim 1, wherein methylation-specific PCR is employed for measuring the DNA methylation.

12. The method according to claim 11, wherein methylated cytosines are converted to uracil using bisulfite.

13. The method according to claim 11, wherein real-time PCR with labeled markers or labeled probes is employed.
